(19) United States
(12) Patent Application Publication
(10) Pub. No.: US 2024/0335576 A1
(43) Pub. Date: Oct. 10, 2024

(54) ULTRAVIOLET TREATMENT APPARATUS AND METHOD

(71) Applicant: LIMESTONE LABS LIMITED, Toronto (CA)

(72) Inventors: THOMAS ALEXANDER MEDAL, VANCOUVER, BRITISH COLUMBIA (CA); CLAIRE DEIRDRE DOBBIN, TORONTO, ONTARIO (CA); ZI-XIANG LIN, NORTH YORK, ONTARIO (CA); MANJUNATH ANAND, TORONTO, ONTARIO (CA); ALYSSA RAMDYAL, SANTA CLARA, CA (US); HARSHRAJ RATNASINH ATODARIA, ETOBICOKE, ONTARIO (CA); NATAN ARIEL MELZER, VAUGHAN, ONTARIO (CA); MATTHEW GARRIN TAYLOR, MISSISSAUGA, ONTARIO (CA)

(21) Appl. No.: 18/573,583

(22) PCT Filed: Jun. 23, 2022
(86) PCT No.: PCT/CA2022/051006

§ 371 Date: Dec. 22, 2023

Related U.S. Application Data
(60) Provisional application No. 63/214,017, filed on Jun. 23, 2021.

(51) Int. Cl. A61L 2/10; A61L 2/24

(52) U.S. Cl. CPC A61L 2/10; A61L 2/24; A61L 2202/11; A61L 2202/121; A61L 2202/14; A61L 2202/16

(57) ABSTRACT

An ultraviolet treatment device, having a housing enclosing a chamber. The chamber has a chamber height along a vertical axis, a chamber width along a transverse axis, and a chamber depth along a longitudinal axis. The chamber depth is less than the chamber width and less than the chamber height, and the chamber depth is less than half of at least one of the chamber width and the chamber height. The device also includes a source of ultraviolet radiation mounted to the housing and arranged to direct the ultraviolet radiation into the chamber, and a lateral wall of the housing having a lateral access opening to the chamber formed in the lateral wall, the lateral access opening having an opening width that is greater than the chamber depth.

### FIELD

The specification relates generally to ultraviolet treatment, and more specifically to an apparatus for and method of treating a handheld object using ultraviolet radiation.

### BACKGROUND

United States Patent Application Publication No. 2021/0128762 to LaPorte et al. (“LaPorte”) purports to disclose a sanitization device comprising primary emitters configured to emit sanitizing electro-optical (EO) radiation into an interior compartment. LaPorte purports to disclose a support member configured to hold and/or secure a target object within the interior compartment of a sanitization device. LaPorte purports to disclose that an end portion of the support member may extend into a secondary compartment comprising a secondary emitter configured to produce external stimulus adapted to excite and recover primary emitters from dormancy. LaPorte purports to disclose that the support member is configured to transmit EO radiation produced by the secondary emitter into the interior compartment to thereby prevent and/or recover from dormancy conditions of the primary emitters. LaPorte purports to disclose that the detection circuitry can detect activation failures of the primary emitters. LaPorte purports to disclose that thermal management circuitry can limit certain critical failures of a sanitization device that result from activation failures of primary emitters.

U.S. Pat. No. 10,064,967 to Nguyen (“Nguyen”) purports to disclose a sterilization enclosure that includes a main body defining a volume and having a movable component configured to move between an access position to allow access to the volume and a restricted position to at least partially enclose the volume. Nguyen purports to disclose that the sterilization enclosure further includes a light source located in the volume and configured to emit light having a frequency that injures pathogens. Nguyen purports to disclose that the sterilization enclosure further includes a switch coupled to the movable component and configured to be in a closed position to allow power to flow to the light source to cause the light source to emit the light into the volume when the movable component is in the closed position.

U.S. Pat. No. 8,296,493 to Engelhardt et al. (“Engelhardt”) purports to disclose a disinfecting docking station for at least one portable electronic device, such as a medical technician's tablet computer, that has at least one recharging connector. Engelhardt purports to disclose that an enclosure is adapted to receive the at least one portable electronic device therein through an openable side that includes a selectively closable door. Engelhardt purports to disclose that each electronic device is exposed to a disinfecting wavelength of light, such as UV light, to disinfect the surface of the device. Engelhardt purports to disclose that the enclosure includes supports that minimally contact each device so that the device will be substantially exposed to the UV light. Engelhardt purports to disclose that a control circuit monitors the exposure time and level, and is programmable to activate the UV light based on pre-set criteria. Engelhardt purports to disclose that the enclosures may be stacked and provide power and network connectivity to each device while docked therein.

### SUMMARY

The following summary is intended to introduce the reader to various aspects of the applicant's teaching, but not to define any invention.

According to some aspects, there is provided an ultraviolet treatment device, comprising a housing enclosing a chamber having a chamber height along a vertical axis, a chamber width along a transverse axis, and a chamber depth along a longitudinal axis, the chamber depth being less than the chamber width and less than the chamber height, wherein the chamber depth is less than half of at least one of the chamber width and the chamber height; a source of ultraviolet radiation mounted to the housing and arranged to direct the ultraviolet radiation into the chamber; a lateral wall of the housing having a lateral access opening to the chamber formed in the lateral wall, the lateral access opening having an opening width that is greater than the chamber depth; and a chamber floor of the housing, the chamber floor or the lateral wall including a retention feature shaped to inhibit sliding of a lower edge of a handheld along the chamber floor towards the lateral access opening when the handheld object is resting in the chamber with the lower edge resting on the chamber floor, the retention feature being transparent to the ultraviolet radiation.

In some examples, the retention feature is selected from the group of a projection from the chamber floor, a projection from the lateral wall, a sloped portion of the chamber floor sloping down away from the lateral access opening, a groove in the chamber floor, a high-friction surface of the chamber floor, and a coarse surface of the chamber floor.

In some examples, the enclosed chamber has a generally rectangular horizontal cross section and the lateral wall is one of a set of lateral walls that includes a pair of broad lateral walls and a pair of narrow lateral walls, and the lateral access opening is formed in one of the broad lateral walls.

In some examples, the housing includes a door moveable between a closed position blocking the lateral access opening and an open position removed from the lateral access opening to provide direct access to the chamber through the lateral access opening for a user to place the handheld object within the chamber with the lower edge resting on the floor.

In some examples, the door is moveable from the closed position to the open position by lifting the door.

In some examples, the door is received in a pocket of the housing when in the open position.

In some examples, the door is a segmented door or includes a flexible panel.

In some examples, the ultraviolet treatment device further comprises a door control system operable to control positioning of the door, the door control system configured to have the open position as a default position of the door when the ultraviolet treatment device is available and not in use.

In some examples, the ultraviolet treatment device further comprises a source of visible light, the source of visible light configured to illuminate the chamber when the door is in the default position.

In some examples, the ultraviolet treatment device further comprises a cycle initiation toggle for use in initiating a cycle.

In some examples, the cycle initiation toggle is selected from the group of a button, a light sensor, a camera, an audio sensor, a touch screen, a door position sensor, and a hygiene dispenser.

In some examples, the cycle initiation toggle includes the button and the button is a multi-function button which is operable to start the cycle if the cycle has not begun and is operable to stop the cycle if the cycle is in progress.

In some examples, the button is an illuminated button.

In some examples, the ultraviolet treatment device is operable to receive power from a power source, and the door is electrically biased to open if the ultraviolet treatment device ceases to receive the power from the power source.

In some examples, the ultraviolet treatment device includes an energy storage device coupled to the power source to detect a loss of power from the power source and operatively coupled to the door to open the door in response to detecting the loss of power from the power source.

In some examples, the ultraviolet treatment device further comprises a screen on an external surface of the ultraviolet treatment device, the screen operable to provide content to a user during a cycle.

In some examples, the ultraviolet treatment device is configured to open the door automatically at the end of the cycle.

In some examples, the housing includes a transparent internal wall forming a wall of the chamber and separating the chamber from the source.

In some examples, the transparent internal wall includes a visibility-enhancing pattern etched into the transparent internal wall or applied to a surface of the transparent internal wall.

According to some aspects, there is provided an ultraviolet treatment device, comprising a housing enclosing a chamber having a chamber height along a vertical axis, a chamber width along a transverse axis, and a chamber depth along a longitudinal axis, the chamber depth being less than the chamber width and less than the chamber height, wherein the chamber depth is less than half of at least one of the chamber width and the chamber height; a source of ultraviolet radiation mounted to the housing and arranged to direct the ultraviolet radiation into the chamber; a lateral wall of the housing having a lateral access opening to the chamber formed in the lateral wall, the lateral access opening having an opening width that is greater than the chamber depth; and a door of the housing, the door moveable between a closed position blocking the lateral access opening and an open position removed from the lateral access opening to provide direct access to the chamber through the lateral access opening, the door moveable from the closed position to the open position by lifting the door.

In some examples, the enclosed chamber has a generally rectangular horizontal cross section and the lateral wall is one of a set of lateral walls that includes a pair of broad lateral walls and a pair of narrow lateral walls, and the lateral access opening is formed in one of the broad lateral walls.

In some examples, the chamber floor or the lateral wall includes at least one retention feature selected from a group including a projection from the chamber floor, a projection from the lateral wall, a sloped portion of the chamber floor sloping down away from the lateral access opening, a groove in the chamber floor, a high-friction surface of the chamber floor, and a coarse surface of the chamber floor, the retention feature being transparent to the ultraviolet radiation.

In some examples, the door is a segmented door or includes a flexible panel.

In some examples, the door is received in a pocket of the housing when in the open position.

In some examples, the ultraviolet treatment device further comprises a door control system operable to control positioning of the door, the door control system configured to have the open position as a default position of the door when the ultraviolet treatment device is available and not in use.

In some examples, the ultraviolet treatment device further comprises a source of visible light, the source of visible light configured to illuminate the chamber when the door is in the default position.

In some examples, the ultraviolet treatment device further comprises a cycle initiation toggle for use in initiating a cycle.

In some examples, the cycle initiation toggle is selected from the group of a button, a light sensor, a camera, an audio sensor, a touch screen, a door position sensor, and a hygiene dispenser.

In some examples, the cycle initiation toggle includes the button and the button is a multi-function button which is operable to start the cycle if the cycle has not begun and is operable to stop the cycle if the cycle is in progress.

In some examples, the button is an illuminated button.

In some examples, the ultraviolet treatment device is operable to receive power from a power source, and the door is electrically biased to open if the ultraviolet treatment device ceases to receive the power from the power source.

In some examples, the ultraviolet treatment device includes an energy storage device coupled to the power source to detect a loss of power from the power source and operatively coupled to the door to open the door in response to detecting the loss of power from the power source.

In some examples, the ultraviolet treatment device further comprises a screen on an external surface of the ultraviolet treatment device, the screen operable to provide content to a user during a cycle.

In some examples, the ultraviolet treatment device is configured to open the door automatically at the end of the cycle.

In some examples, the housing includes a transparent internal wall forming a wall of the chamber and separating the chamber from the source.

In some examples, the transparent internal wall includes a visibility-enhancing pattern roughed into the transparent internal wall or applied to a surface of the transparent internal wall.

According to some aspects, there is provided a method of treating a handheld object, the handheld object having a broad planar surface and an adjacent lower edge, the method comprising inserting the handheld object into a chamber of an ultraviolet treatment device through a lateral access opening; positioning the handheld object in the chamber with the broad planar surface of the handheld object directed towards the lateral access opening and the lower edge of the handheld device resting on a floor of the chamber; directing, while the handheld object is in the chamber with the broad planar surface of the handheld object directed towards the lateral access opening and the lower edge of the handheld object resting on the floor, ultraviolet radiation towards the handheld object from an ultraviolet radiation source of the ultraviolet treatment device; and removing the handheld object from the chamber after directing ultraviolet radiation towards the handheld object.

In some examples, the method further comprises, between positioning the handheld object in the chamber and directing ultraviolet radiation towards the handheld object, closing a door of the ultraviolet treatment device to block the lateral access opening by lowering the door.

In some examples, the door is a segmented door or includes a flexible panel, and lowering the door includes drawing the door down out of a pocket of a housing of the ultraviolet treatment device.

In some examples, the method further comprises, between directing ultraviolet radiation towards the handheld object and removing the handheld object from the chamber, opening a door of the ultraviolet treatment device to clear the lateral access opening by lifting the door.

In some examples, the door is a segmented door or flexible panel, and lifting the door includes moving the door into a pocket of a housing of the ultraviolet treatment device.

In some examples, positioning the handheld object in the chamber includes arranging the lower edge against a retention feature on the floor of the chamber or on the lateral wall, the retention feature shaped to inhibit sliding of the lower edge along the floor towards the lateral access opening, the retention feature transparent to the ultraviolet radiation.

In some examples, the retention feature is selected from a group including a projection from the chamber floor, a projection from the lateral wall, and a sloped portion of the chamber floor sloping down away from the lateral access opening, a groove in the chamber floor, a high-friction surface of the chamber floor, and a coarse surface of the chamber floor.

In some examples, directing ultraviolet radiation towards the handheld object includes triggering a cycle initiation toggle of the ultraviolet treatment device.

In some examples, the cycle initiation toggle is selected from the group of a button, a light sensor, a camera, an audio sensor, a touch screen, a door position sensor, and a hygiene dispenser.

In some examples, the cycle initiation toggle includes the button and the button is a multi-function button which is operable to start the cycle if the cycle has not begun and is operable to stop the cycle if the cycle is in progress.

In some examples, the button is an illuminated button.

In some examples, the ultraviolet treatment device is operable to receive power from a power source, and the door is electrically biased to open if the ultraviolet treatment device ceases to receive the power from the power source.

In some examples, the ultraviolet treatment device includes an energy storage device coupled to the power source to detect a loss of power from the power source and operatively coupled to the door to open the door in response to detecting the loss of power from the power source.

In some examples, the ultraviolet treatment device includes a screen on an external surface of the ultraviolet treatment device, the screen operable to provide content to a user during a cycle.

In some examples, the handheld object is a personal computing device.

In some examples, the ultraviolet treatment device is configured to open the door automatically after directing ultraviolet radiation towards the handheld object.

In some examples, the ultraviolet treatment device includes a transparent internal wall forming a wall of the chamber and separating the chamber from the source.

In some examples, the transparent internal wall includes a visibility-enhancing pattern roughed into the transparent internal wall or applied to a surface of the transparent internal wall.

### BRIEF DESCRIPTION OF THE DRAWINGS

The drawings included herewith are for illustrating various examples of articles, methods, and apparatuses of the present specification and are not intended to limit the scope of what is taught in any way. In the drawings:

FIG. 1 is a perspective view of an ultraviolet treatment device;

FIG. 2 is a perspective view of the ultraviolet treatment device of FIG. 1 with a first handheld object received inside;

FIG. 3 is a perspective view of the ultraviolet treatment device of FIG. 1 with second and third handheld objects received inside;

FIG. 4 is a perspective cross sectional view of the ultraviolet treatment device of FIG. 1 with the first handheld object received inside;

FIG. 5 is a perspective view of the ultraviolet treatment device of FIG. 1 with a door in a closed position;

FIG. 6 is a perspective cross sectional view of the ultraviolet treatment device of FIG. 1 with the door in the closed position;

FIG. 7 is a perspective cross sectional view of the ultraviolet treatment device of FIG. 1 with the door in an open position;

FIG. 8 is a perspective cross sectional view of a portion of the ultraviolet treatment device of FIG. 1;

FIG. 9 is an exploded view of the ultraviolet treatment device of FIG. 1;

FIG. 10 is a perspective cross sectional view of the ultraviolet treatment device of FIG. 1 with a tray or liner removed to show an ultraviolet source;

FIG. 11 is a perspective view of the ultraviolet treatment device of FIG. 1 with a visibility-enhancing pattern;

FIG. 12 is a perspective view of a cycle initiation toggle of the ultraviolet treatment device of FIG. 1;

FIG. 13 is a top view of the ultraviolet treatment device of FIG. 1;

FIG. 14 is a front view of the ultraviolet treatment device of FIG. 1;

FIG. 15 is a rear view of the ultraviolet treatment device of FIG. 1;

FIG. 16 Is a bottom view of the ultraviolet treatment device of FIG. 1;

FIG. 17 is a rear perspective view of the ultraviolet treatment device of FIG. 1 with a first set of brackets and a first mounting plate;

FIG. 18 is a rear perspective view of the ultraviolet treatment device of FIG. 1 with a second bracket and a second mounting plate;

FIG. 19 is a front perspective view of the second bracket;

FIG. 20 is a rear perspective view of the ultraviolet treatment device of FIG. 17 with the second mounting plate received in the second bracket;

FIG. 21 is a perspective view of the ultraviolet treatment device of FIG. 1 on a table stand;

FIG. 22 is a front perspective view of the ultraviolet treatment device of FIG. 1 on a floor stand;

FIG. 23 is a rear perspective view of the ultraviolet treatment device of FIG. 1 on the floor stand; and,

FIG. 24 is a rear perspective view of the ultraviolet treatment device of FIG. 1 on a lean stand; and,

FIG. 25 is a front perspective view of the ultraviolet treatment device of FIG. 1 on the lean stand;

FIG. 26 is a flow chart of a method of treating a handheld object using an ultraviolet treatment device.

### DETAILED DESCRIPTION

Various apparatuses or processes will be described below to provide an example of an embodiment of each claimed invention. No embodiment described below limits any claimed invention and any claimed invention may cover processes or apparatuses that differ from those described below. The claimed inventions are not limited to apparatuses or processes having all of the features of any one apparatus or process described below or to features common to multiple or all of the apparatuses or process described below. It is possible that an apparatus or process described below is not an embodiment of any claimed invention. Any invention disclosed in an apparatus or process described below that is not claimed in this document may be the subject matter of another protective instrument, for example, a continuing patent application, and the applicants, inventors or owners do not intend to abandon, disclaim, or dedicate to the public any such invention by its disclosure in this document.

Although method steps may be described (in the disclosure and/or in the claims) in a sequential order, such methods may be configured to work in alternate orders. In other words, any sequence or order of steps that may be described does not necessarily indicate a requirement that the steps be performed in that order. The steps of methods described herein may be performed in any order that is practical. Further, some steps may be performed simultaneously.

Furthermore, it will be appreciated that for simplicity and clarity of illustration, where considered appropriate, reference numerals may be repeated among the figures to indicate corresponding or analogous elements. In addition, numerous specific details are set forth in order to provide a thorough understanding of the example embodiments described herein. However, it will be understood by those of ordinary skill in the art that the examples described herein may be practiced without these specific details. In other instances, well-known methods, procedures, and components have not been described in detail so as not to obscure the examples described herein.

As used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

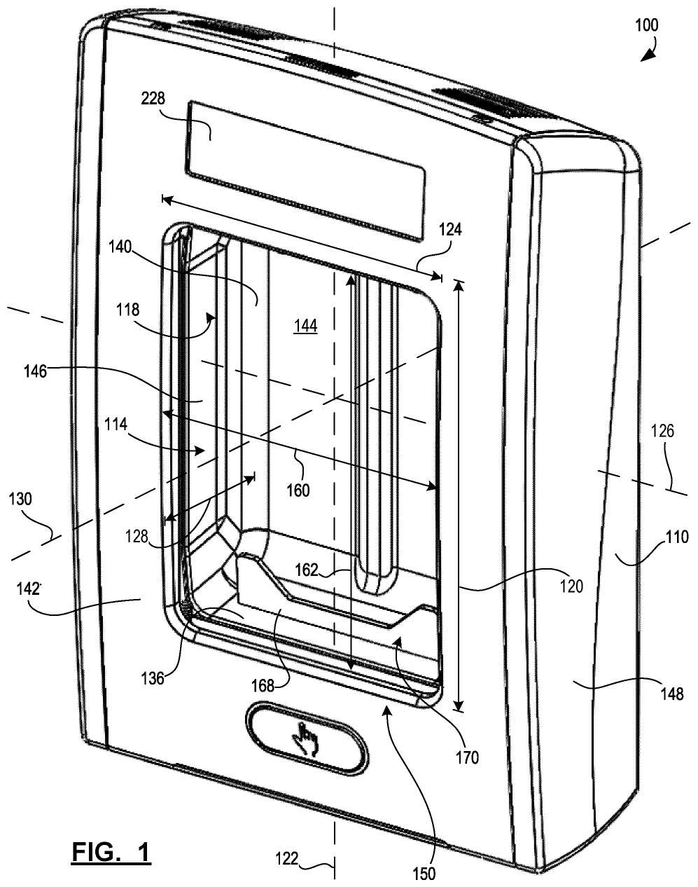

Referring to FIG. 1, illustrated is an example of an ultraviolet treatment device 100. The ultraviolet treatment device 100 is operable to treat an object that is placed within the ultraviolet treatment device 100 by directing ultraviolet radiation at the object.

The ultraviolet treatment device 100 may be a germicidal device, disinfection device, and/or a sanitization device. In other words, the ultraviolet treatment device 100 may be used to disinfect or sanitize an object received within the ultraviolet treatment device 100. Sanitizing the object may include killing germs (viruses and/or bacteria) on the object, such as killing at least 90% of the population, at least 99% of the population, at least 99.99% of the population, or at least 99.9999% of the population.

The ultraviolet treatment device 100 includes a housing 110. The housing 110 may be opaque to ultraviolet radiation. For example, the housing 110 may be made of and/or include plastic and/or metal panels. The housing 110 encloses an internal chamber 114. The housing 110 includes a set of walls 118 enclosing the chamber. The chamber 114 has a chamber height 120 along a vertical axis 122, a chamber width 124 along a transverse axis 126, and a chamber depth 128 along a longitudinal axis 130.

As in the illustrated example, the set of walls 118 may include a floor 136, a ceiling 138 (FIG. 4), and a lateral wall 140 extending between the floor 136 and the ceiling 138. The chamber 114 may be generally cuboid in shape, with the lateral wall 140 including a front wall 142, a rear wall 144, a right side wall 146, and a left side wall 148. However, it will be understood that the chamber 114 may also have another shape (e.g., spherical, cylindrical or amorphous) in other examples.

The chamber 114 may be shaped to receive a particular type of handheld object. In some examples, the chamber depth 128 is relatively small to facilitate use with a handheld object having a relatively thin depth. The chamber depth 128 is less than the chamber height 120 and is less than the chamber width 124. The chamber depth 128 may be less than three quarters, less than half, less than a quarter, or less than a fifth of the chamber height 120. The chamber depth 128 may be less than three quarters, less than half, less than a quarter, or less than a fifth of the chamber width 124.

A relatively small chamber depth 128 may facilitate receiving a handheld object having a broad and planar front face, as described further below. A relatively small chamber depth 128 may allow a user to easily position a handheld object within the chamber 114 (e.g., in an upright position resting on a narrow side or edge of the handheld object) and/or retrieve the object. For example, the user may be able to lean the handheld object against the rear wall 144 with an edge of the handheld object on the floor 136, and may do so without reaching deep into the chamber 114, touching a wall of the chamber 114, laying the handheld object down, and/or sliding the handheld object across a wall of the chamber (e.g., the floor 136). A relatively small chamber depth 128 may facilitate positioning a handheld object in the chamber in an upright position (i.e., resting with an edge of the handheld object on a floor 136 of the chamber 114).

In some examples, the chamber 114 is a vertically oriented chamber (i.e., a portrait configuration) with a height that is greater than the width (e.g., the height is more than 1.5 times or more than 2 times the size of the width). In some examples, the chamber 114 may be a horizontally oriented chamber (i.e., a landscape configuration) with a width that is greater than the height (e.g., the width is more than 1.5 time or more than 2 times the height). In some examples, the chamber 114 has a chamber height 120 equal to the chamber width 124.

The ultraviolet treatment device 100 also includes a source of ultraviolet radiation 230 (e.g., one or more lamps, such as light emitting diodes, operable to emit ultraviolet radiation, as discussed further below), as will be discussed further below. The source of ultraviolet radiation is arranged to irradiate an object that is placed in the chamber 114.

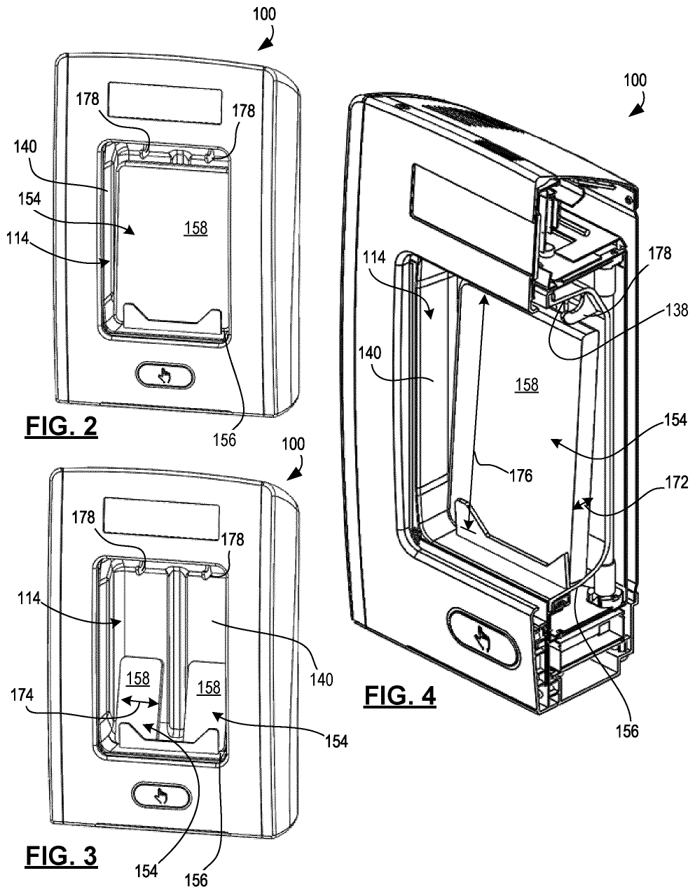

The handheld object may be inserted into the chamber 114 through a lateral access opening 150 in the lateral wall 140 of the housing 110. The lateral access opening 150 may provide direct access to the chamber 114 in which the handheld object would be irradiated. The lateral access opening 150 may be shaped to allow a user to place the handheld object 154 (FIGS. 2 to 4) directly in the chamber with a lower edge 156 resting on a floor of the chamber 114 and a broad face 158 of the handheld object 154 directed at (e.g., facing) the lateral access opening 150.

The lateral access opening 150 may be in a broad face of the housing 110. For example, the enclosed chamber 114 may have a generally rectangular horizontal cross section, and the lateral wall 140 in which the lateral access opening 150 is formed may be one of the broad walls of a set of lateral walls 140 that includes a pair of broad lateral walls (e.g., front and back walls 142, 144) and a pair of narrow lateral walls (e.g., side walls 146, 148). As in the illustrated example, the lateral access opening 150 may be in a front wall 142. In some examples, the lateral access opening has a width 160 and a height 162. The width 160 may be less than or equal to the chamber width 124. The height 162 may be greater than or equal to the chamber height 120. The width 160 is greater than the chamber depth 128 (i.e., the opening 150 is in a broad wall). A wide lateral access opening 150 may facilitate easy insertion of the handheld object 154, particularly if the handheld object 154 has a broad width.

A device 100 having the access opening 150 in a broad lateral wall and having a vertically oriented chamber with a short depth may facilitate use, however the handheld object 154 may be at risk of falling out of the lateral access opening 150. In some examples, since the lateral access opening 150 provides direct access to a narrow chamber 114, a retention feature or member 170 is provided to inhibit the handheld object from falling out through the lateral access opening. The chamber floor 136 and/or the lateral wall 140 of the housing includes the retention feature or member 170. For example, the retention feature 170 may be part of and/or be secured to and extend from one or more of the side walls 146, 148 or front or back walls 142, 144 and/or from the floor 136.

In the illustrated example, a single retention feature or member 170 is shown and it extends from the floor 136, however in other examples more than one retention feature or member 170 may be used on the floor 136 and/or the lateral wall 140.

The retention feature 170, the chamber floor 136, and/or the lateral wall 140 may be transparent to ultraviolet and/or visible radiation. In some embodiments, the retention feature 170 is transparent to ultraviolet radiation. A retention feature 170 that is transparent to ultraviolet radiation allows a user to make use of the floor and retention feature to support an object, since the retention feature does not block the ultraviolet light from reaching the object. Using the floor and retention feature to support the object allows the chamber 114 to be smaller and/or hold more or different objects than if the objects needed to be suspended in the chamber 114.

The retention feature 170 is shaped to inhibit sliding of the lower edge of the handheld object along the chamber floor 136 towards the lateral access opening 150 when the handheld object is resting in the chamber 114 with the lower edge resting on the chamber floor 136. For example, the retention feature 170 may be a projection (e.g., a lip or ridge 168, as in the illustrated example) from the floor 136 and/or side wall 146, 148 into the chamber 114. In some examples, the retention feature 170 is selected from the group of a projection from the chamber floor 136, a projection from the lateral wall 140, a sloped portion of the chamber floor 136 sloping down away from the lateral access opening 150, a groove in the chamber floor 136, a high-friction surface of the chamber floor 136, and a coarse surface of the chamber floor 136. As exemplified, in some embodiments the retention feature 170 is spaced from the lateral access opening (e.g., by at least 10%, 25% or 50% of the depth of the chamber 114). As exemplified, in some embodiments the retention feature 170 is a projection spaced at least 25% of the depth of the chamber 114 from the lateral access opening.

Referring now to FIGS. 2 to 4, in some examples, the handheld object 154 has a broad and/or planar front face 158, as indicated above. The handheld object 154 may also have a lower edge 156 upon which the handheld object 154 may rest when the handheld object 154 is within the chamber 114 (e.g., when the handheld object 154 is leaning against a lateral wall 140 of the chamber 114).

In some examples, the handheld object 154 is a handheld computing device, such as a smartphone or tablet. The handheld computing device may be a generally cuboid device. In some examples, the handheld computing device 154 has a depth 172 that is relatively thin comparted to the width 174 and height 176 of the front face 158 (i.e., the screen) of the handheld computing device 154. For example, the depth 172 may be less than ⅕th or less than 1/10th the width 174 and/or height 176 of the front face 158.

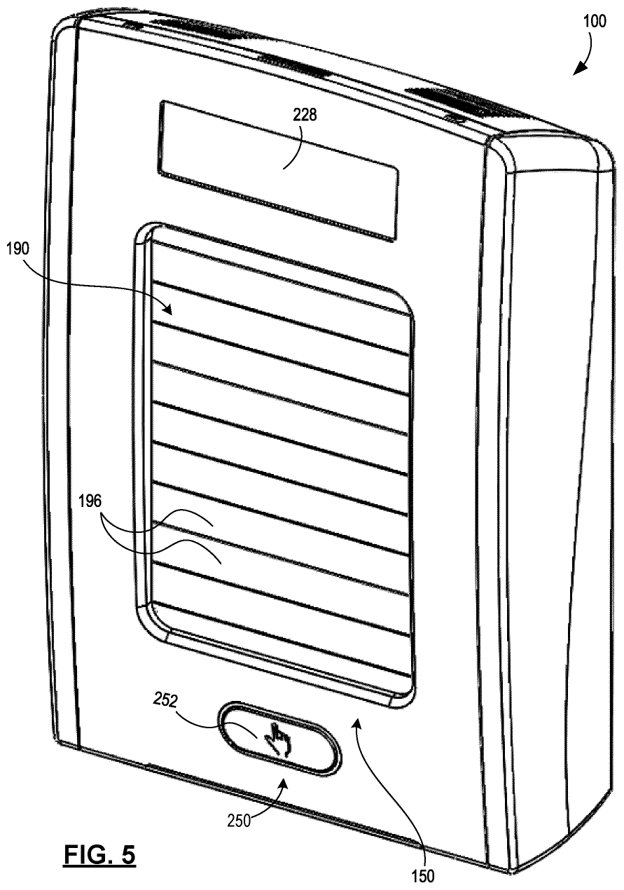

The chamber 114 may be shaped to hold a handheld computing device (e.g., an iPad™ or similar tablet device). However, it will be appreciated that this does not preclude use of the chamber 114 to treat one or more other objects (e.g., clip-boards, keys, masks, or stethoscopes). For example, the ultraviolet treatment device 100 may be used to treat one or more smartphones as shown in FIG. 3, one or more tablets, one or more sets of keys, and/or one or more masks. For example, the ultraviolet treatment device 100 may include a hook 178 from which a mask, set of keys, or stethoscope may be hung above and/or beside one or more other device such as a smartphone and/or tablet.

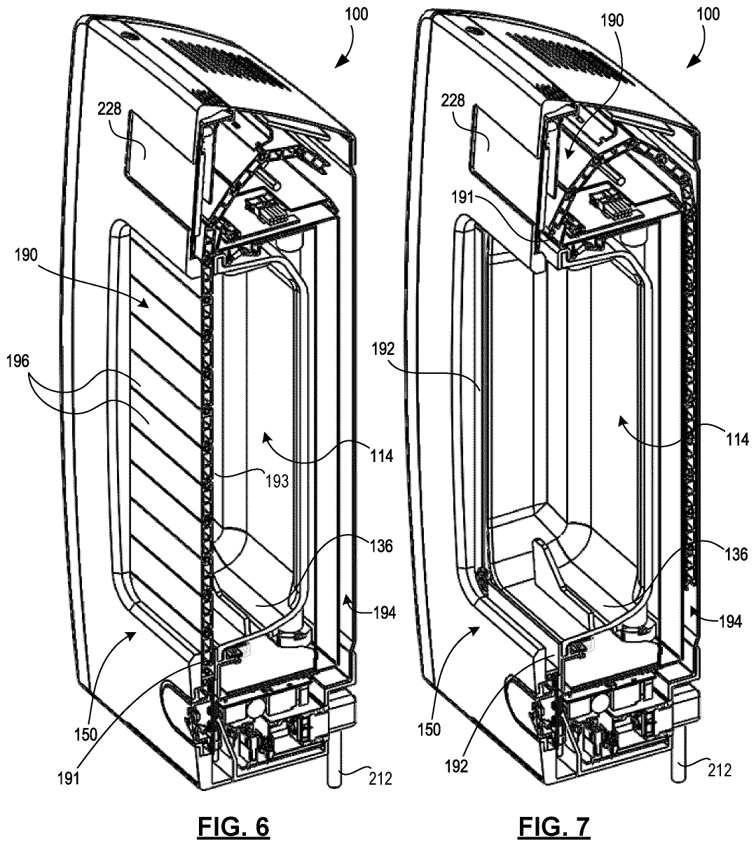

Referring now to FIG. 5, the ultraviolet treatment device 100 (e.g., the housing 110) includes a door 190. The lateral access opening 150 can be closed by the door 190. The door 190 is opaque to ultraviolet radiation (e.g., including plastic or metal panels) so that radiation that may be harmful to humans can be blocked. The door 190 may completely close off the lateral access opening 150 to prevent radiation from leaking out through the lateral access opening 150. In some examples, a surface of the door 190 is reflective. The door 190 may include a surface that is exposed to the chamber 114 when the door 190 is closed (e.g., a rear surface 193 of the door 190, as shown in FIG. 6), and that surface may be reflective.

Referring now to FIGS. 6 and 7, the door 190 is moveable between a closed position (e.g., FIG. 6) blocking the lateral access opening 150 and an open position (e.g., FIG. 7) removed at least partially from the lateral access opening 150 to provide direct access to the chamber 114 through the lateral access opening 150 for a user to place the handheld object 154 within the chamber 114 with the lower edge 156 resting on the floor 136. In some examples, one or more peripheral edges 191 of the door 190 rests in a groove or slot or pocket 192 of the housing 110. For example, as in the illustrated example, the groove 192 may extend around a large portion (e.g., 3 of the 4 sides) of the perimeter of the lateral access opening 150, and the door 190 can rest in the groove 192 around the perimeter of the lateral access opening 150 when the door is in the closed position.

The door 190 may be opened and/or closed in various ways. In some examples, the door 190 is moveable from the closed position to the open position by lowering the door 190 and/or lifting the door 190. In some examples, the door 190 is received in a pocket 194 of the housing 110 when in the open position. For example, in the illustrated example, the door 190 is moveable from the closed position to the open position by lifting the door 190 to move the door 190 into the pocket 194. The door 190 is configured to be moved by a drive system (e.g., an electronic drive system 198 controlled by a control unit 210, as shown in the example of FIG. 9), but may also be manually pushed up if need be. Lowering and/or lifting the door may facilitate use, by keeping the door out of the way of users. For example, lifting the door may facilitate use by users who may be accessing the ultraviolet treatment device 100 from a position that is below the ultraviolet treatment device 100. A user in a wheelchair or a child, for example, may access the ultraviolet treatment device 100 from a lower position (e.g., if the ultraviolet treatment device 100 is positioned on a wall or counter).

While it will be appreciated that many types of doors may be used, in some examples the door 190 has an adjustable shape. This may facilitate moving the door 190 between open and closed positions. For example, the door 190 may include a flexible panel. In some examples, as in the illustrated example, the door 190 is a segmented door (e.g., a tambour-style door). The segments 196 are secured together as a panel with flexibility in at least one dimension. In some examples, the door 190 is configured to be adjusted in shape when moved between the open and closed positions. This may facilitate receiving the door 190 in the housing 110 in the open position (e.g., in the pocket 194).

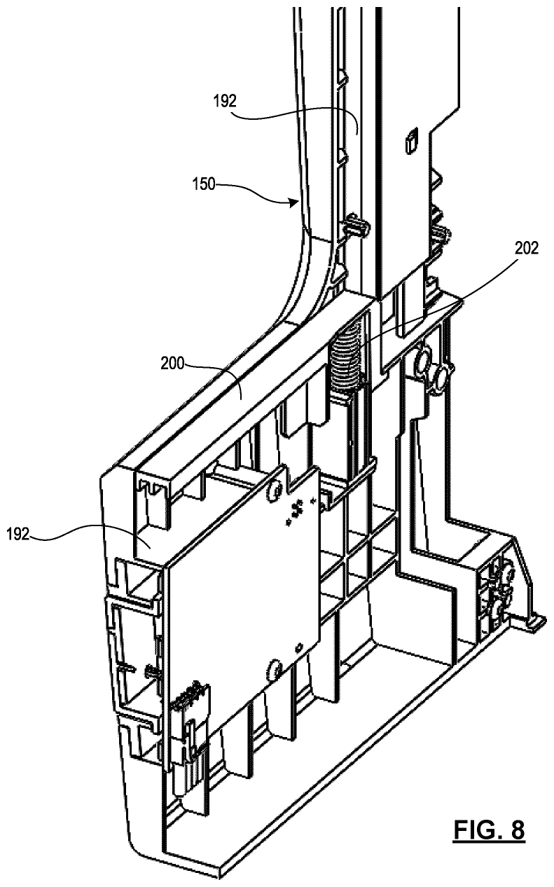

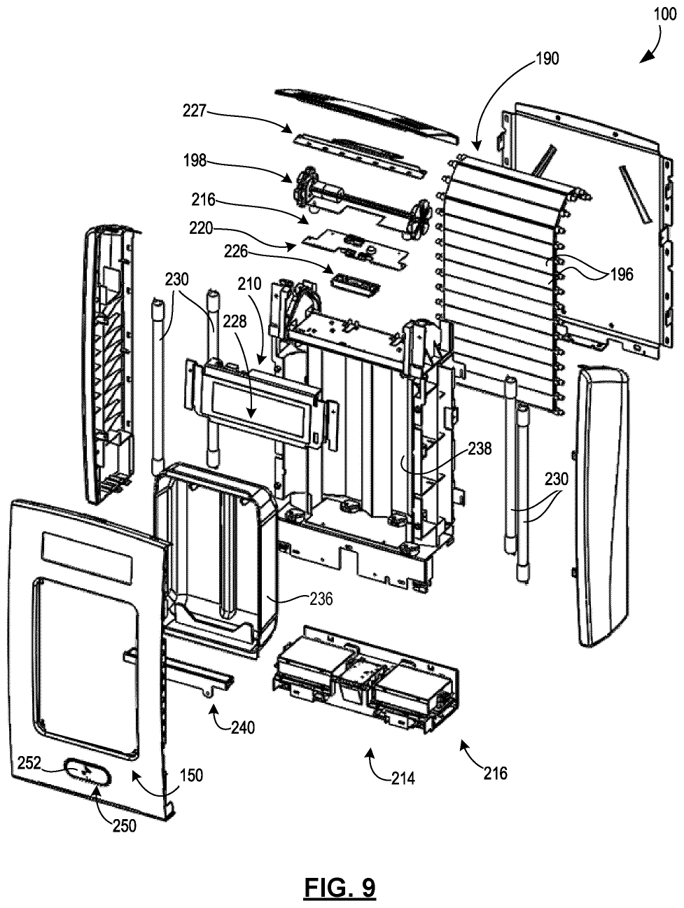

Referring now to FIG. 8, the ultraviolet treatment device 100 may include a cover 200 over at least a portion of the groove (e.g., the bottom portion at the bottom of the opening 150, as illustrated) to prevent objects from falling into and/or getting stuck in the groove 192. The cover 200 may be a moveable floor, and may be moveable between an extended (e.g., raised) position in which it blocks off at least a portion of the depth of the groove 192 and a retracted (e.g., lowered) position in which it is moved such that the portion of the depth of the groove 192 is no longer blocked. For example, the cover 200 may be moved farther into (e.g., down into) the groove to allow the door 190 to occupy the portion of the depth of the groove 192.

In some examples, the door 190 may move the cover 200 to the retracted position. The cover 200 may be biased to the extended position (e.g., via springs 202). The force required to move the cover 200 may be selected to be less than the force provided by the door 190 when the door 190 moves from the open position to the closed position. While the cover 200 is shown as a moveable floor over a horizontally extending lower portion of the groove 192 in the illustrated example, it is to be understood that the cover 200 may also or alternatively cover a vertical or horizontal side or top portion of the groove 192 in other examples.

The cover 200 may activate sensors or toggles or switches (e.g., a door position sensor that needs to be activated before a control system will increase power to an ultraviolet source to begin the radiation portion of a cycle) when moved by the door 190 into the retracted position. The sensors or toggles or switches may be inaccessible to the user behind the cover 200, which may improve user safety (e.g., by preventing the user from directly activating these switches).

Referring now to FIG. 9, in some examples, the ultraviolet treatment device 100 includes a control system 210 (e.g., a control system exclusively to operate the door, or a control system to operate multiple components of the device 100) operable to control the position of the door 190. The door control system 210 may include a processor, a data storage device (e.g., to store programed position information), and a power supply. The power supply may be an onboard power supply (e.g., batteries, capacitors, etc.) and/or a power coupling (e.g., a power cord) to receive power from an external source (e.g., residential current). In some examples, the ultraviolet treatment device 100 is configured to be powered by the power coupling 212 (FIGS. 6 and 7) as a primary power supply. The onboard power supply 214 may be included in addition to the power coupling to provide a backup power supply 216. Additionally or alternatively, a backup on-board power supply 216 may be integrated with a module for a visible light source 220 (described below).

In some examples, the ultraviolet treatment device 100 (e.g., the control system 210 and/or backup power supply 216) may be configured to detect a loss of power from the power coupling (e.g., if the residential current is no longer available), and respond by initiating a loss of power protocol. The loss of power protocol may include ending any ongoing cycles and/or opening the door 190 (e.g., opening the door 190 at least an inch or at least two inches to enable a user to pull the door 190 open further). The backup power supply may be used to open the door as part of the loss of power protocol (i.e., the door may be electrically biased to open if the ultraviolet treatment device ceases to receive power from the main power source). The backup power supply (e.g., onboard power supply) may be coupled to the main power supply (e.g., the power coupling) to be charged by the main power supply.

The door control system 210 may be configured to have the open position as a default position of the door when the ultraviolet treatment device is available and not in use (i.e., coupled to a main power supply and set up for use but not currently running a cycle). While in some examples the default position when not in use may be closed, having the default position as the open position may encourage user interaction with the ultraviolet treatment device 100 and/or decrease the total cycle or engagement time required.

In some examples, the ultraviolet treatment device 100 includes a visible light source 220 (e.g., one or more lamps, such as light emitting diodes) operable to illuminate the interior of the chamber 114. The visible light source 220 may be, as in the illustrated example, in a wall of the chamber 114 (e.g., the ceiling 138) and arranged to emit visible light into the chamber. In some examples, the visible light source 220 is configured to illuminate the interior of the chamber 114 when the door 190 is open and/or partially open (e.g., in the default position). For example, the control system 210 may be operatively coupled to the visible light source 220 to control operation of the visible light source 220, and may be configured to increase power to the visible light source 220 when the door 190 is open and/or decrease power to the visible light source 220 when the door 190 is closed. Lighting the chamber 114 may encourage user interaction with the chamber 114. For example, a user may be more likely to notice and/or consider using the ultraviolet treatment device 100 if the chamber is illuminated.

The ultraviolet treatment device 100 may also include a camera 226. The camera 226 is arranged to image an interior of the chamber 114. The camera 226 may be arranged to image the object 154 when the object 154 is positioned in the chamber 114. The camera 226 may be configured to capture one or more images of the chamber 114 and/or the object 154 in the chamber 114 when the door 190 is closed. The camera 226 may be communicatively coupled to a control system (e.g., the control system 210), and the control system may be configured to direct the camera 226 to take an image or series of images before, during, and/or after the door 190 is shut and/or a cycle is initiated. The output of the camera 226 may be passed to the control system and/or sent to another device (e.g., a remote server).

The ultraviolet treatment device 100 may include an external communications link, such as a wired connection or a wireless transmitter and/or receiver (e.g., a GSM™, Wi-Fi™, and/or Bluetooth™ antenna mounted on antenna mount 227). A communications link may be used to send data to an external device (e.g., usage data, performance data, or camera output) and/or receive instructions from an external device (e.g., to initiate a cycle, initiate advertising or marketing on a screen of the device 100 or an exterior screen, or adjust the settings of the control system 210 such as to change the duration of an ultraviolet radiation phase of a cycle).

The ultraviolet treatment device 100 may also include a screen 228. The screen 228 is and/or is on an exterior surface of the housing 110. The screen 228 is provided to present content to a user. The screen 228 may be configured to present an image or series of images to entertain a user during a cycle (e.g., news or an information video). The screen 228 may alternatively or additionally be configured to present an image or series of images from the camera 226. For example, the screen 228 may display the output of the camera 226 in real time, at least when the door 190 is closed. This may help to reassure users that the object 154 placed in the chamber 114 is being handled with care.

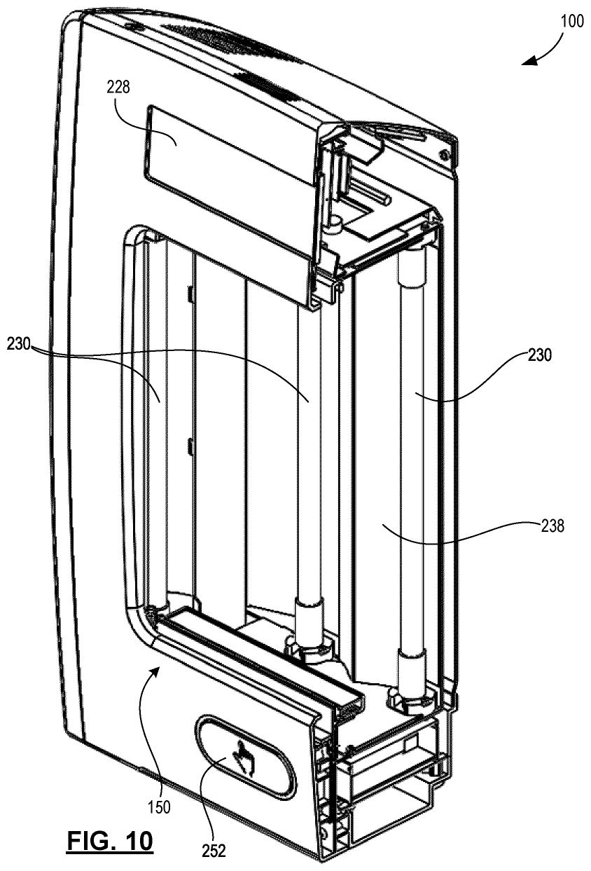

Referring now to FIG. 10, the ultraviolet treatment device 100 includes an ultraviolet radiation source 230. The ultraviolet radiation source 230 may be one or more lamps (e.g., light emitting diodes) operable to emit ultraviolet radiation. The ultraviolet radiation source 230 is arranged to irradiate an object in the chamber.

In some examples, as in the illustrated example, the ultraviolet radiation source 230 is a plurality of sources arranged around the chamber 114. The sources 230 may be separated from the chamber by a tray or liner 236 (FIG. 9). For example, the tray or liner 236 (e.g., a transparent tray) may protect the sources 230 from tampering and/or damage. A reflective surface 238 (e.g., a surface on a reflector) is arranged behind the ultraviolet radiation sources 230 to reflect radiation that is emitted away from the chamber 114 back towards the chamber 114. The reflective surface 238 and/or tray or liner 236 may also be shaped to spread the radiation throughout the chamber 114 and/or towards the chamber 114 at different angles than initially emitted by the sources 230.

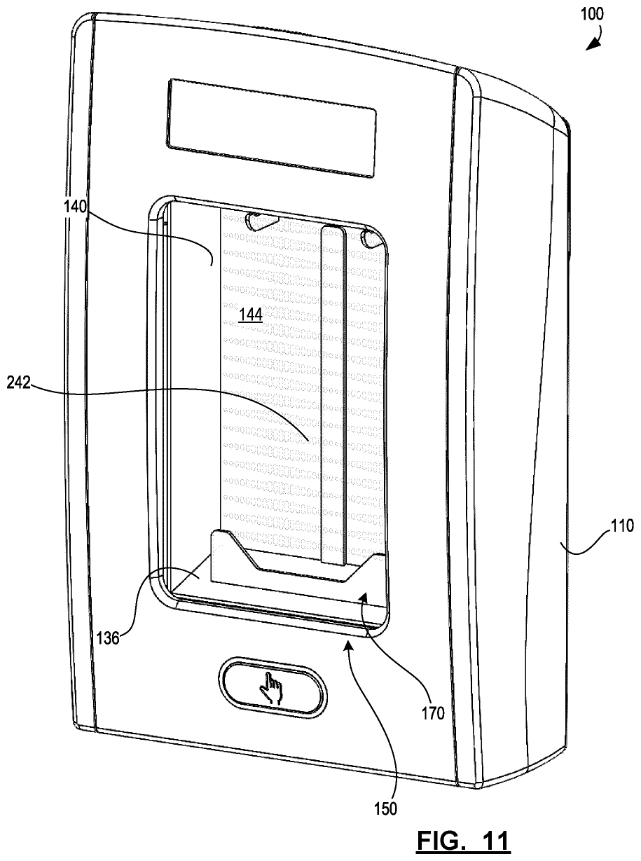

Referring now to FIG. 11, in some embodiments the device 100 includes an interior transparent wall or walls that are transparent to ultraviolet radiation. In some embodiments, the transparent wall or walls are also or alternatively transparent to visible radiation. A wall or walls transparent to visible radiation may be difficult for a user of the device 100 to see, particularly when the transparent wall or walls are arranged in front of reflective surfaces.

As exemplified in FIG. 11, the device 100 may include a visibility-enhancing pattern 242 integrated into a wall. As exemplified, the wall may be a rear wall 144. The wall with the pattern may separate the chamber 114 from the ultraviolet source 230 and/or a reflective surface within the device 100. The visibility-enhancing pattern may be within or applied to a wall. For example, the pattern 242 may be a frosted or roughed finish, such as etched (e.g., by acid) or abrasively blasted (e.g., sand blasting) into the wall (e.g., a glass or plastic wall). In another example, the pattern 242 may be applied to an outer surface of the wall (e.g., a layer overlying the outer surface, such as a sticker or paint applied to the outer surface). In some embodiments, the visibility-enhancing pattern 242 is a portion of the wall having a reduced-transparency compared to the rest of the wall.

Referring again to FIG. 9, the ultraviolet treatment device 100 may also include an ultraviolet source control system communicatively coupled to the ultraviolet source 230 to control operation of the ultraviolet source 230. A common control system 210 may control the ultraviolet source 230 and the door 190, as in the illustrated example. The control system 210 may be configured to prevent operation of the source 230 when the door 190 is open. The control system 210 may be communicative coupled to a sensor 240 to determine the position of the door 190. For example, the sensor 240 may be a Hall effect sensor or a line break sensor (e.g., an infrared break-beam sensor) arranged to sense a break in a beam. As in the illustrated example, the sensor may be arranged at an end or side of the lateral access opening 150 at which the door 190 would arrive last when moving from the open position to the closed position. The line break sensor may be arranged such that a break sensed by the line break sensor indicates that the door 190 is closed. As mentioned above, in some examples, the sensors are below the cover 200 and are activated by the cover 200 being in the retracted position.

The device 100 may also include a sensor system to detect that the opening 150 is blocked by an outside device such as a hand or an object that is too large for the chamber. For example, the camera 226 may cooperate with a non-ultraviolet radiation source (e.g., infrared radiation source) to detect that the opening 150 is blocked. As in the illustrated example, the camera 226 may be adjacent the top of the opening 150 and the source may be at another end or side (e.g., the bottom end).

The control system 210 may be configured to increase power to the source 230 when the door 190 is closed and a cycle has been triggered.

The ultraviolet treatment device 100 includes a cycle initiation toggle 250 for use in initiating or triggering a cycle of the ultraviolet treatment device 100. For example, the control system 210 may be communicative coupled to the cycle initiation toggle 250 to receive an indication of a cycle initiation from the toggle 250 (e.g., a button or a timer counting down from a sensed event such as a depositing of a device in the chamber), and may be configured to respond to the indication by initiating a cycle. The cycle includes, as a radiation phase of the cycle, operating (e.g., increasing power to) the ultraviolet light source 230 to emit a predetermined level of radiation, for a predetermined period of time, and/or a predetermined dosage. In some examples, the cycle includes monitoring the dosage that has been delivered (e.g., via a sensor in the chamber 114 measuring radiation received and/or by measuring an output of a radiation source 230), and the cycle may end when a predetermined dosage is delivered. The predetermined dosage may be more than 10 mJ/cm², more than 50 mJ/cm², more than 100 mJ/cm², more than 200 mJ/cm², more than 300 mJ/cm², or more than 1000 mJ/cm². This predetermined period of time may be set by the user or preset. The cycle duration may be variable (i.e., able to be reset), and may be changed by a user, e.g., via a toggle or interface on the device 100 or via an external device as described above. For example, the predetermined period of time may be between 1 second 10 minutes, between 1 second and 180 seconds, or between 5 seconds and 120 seconds. The cycle may consist of the radiation phase, or the cycle may include a further phase (e.g., one or more further phases). In some examples, the cycle includes closing the door 190, e.g., prior to powering up the ultraviolet light source 230 to emit the predetermined level of radiation. In some examples, the cycle also or alternatively includes opening the door 190 and/or returning the door 190 to the default position after the predetermined period of time.

The cycle initiation toggle 250 may be provided as and/or on an exterior surface of the housing 110. The cycle initiation toggle 250 may also be used to initiate another action (e.g., a multifunction toggle). For example, a cycle initiation toggle 250 may be used to start a cycle, stop a cycle, and/or change a cycle. In some examples, the cycle initiation toggle 250 is selected from the group of a button (e.g., an illuminated button), a light sensor, a camera, an audio sensor, a touch screen, a door position sensor operable to sense a position of the door 190 (e.g., to initiate a cycle in response to a closing of the door 190), and a hygiene dispenser. Triggering the cycle initiation toggle 250 may initiate an action. For example, a control system (e.g., control system 210) may be communicatively coupled to the cycle initiation toggle 250 to receive a signal indicating that the toggle 250 has been triggered, and may respond by initiating the action.

For example, a button or touchscreen may be arranged on an exterior surface of the housing 110 to be triggered by being pressed and/or touched by a user. In another example, a sensor such as a light sensor, camera, or audio sensor may be arranged on an exterior surface of the housing 110 and may be configured to be triggered by motion and/or sound (e.g., motion or sound within 10 meters of the ultraviolet treatment device 100). A door position sensor may be mounted to the housing 110 (e.g., inside the chamber 114, inside the lateral access opening 150, or on an exterior surface) and arranged to sense when a door is moved to a closed position (e.g., shut by a user) and/or shut automatically in response to a command from a user.

A hygiene dispenser (e.g., dispenser 211 of FIG. 18) may also be used as a cycle initiation toggle. The hygiene dispenser may be communicatively coupled to a control system (e.g., the control system 210) and triggered by a dispensing action of the hygiene dispenser. For example, a dispensing action may be a user activating a touchless dispenser or depressing a pump of the dispenser to pump out a hand sanitizer (e.g., for personal use), or a user activating a towelette or wipe dispenser to dispense a towelette or wipe.

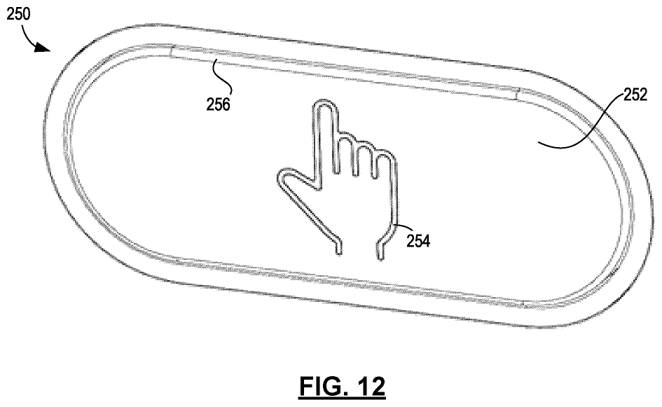

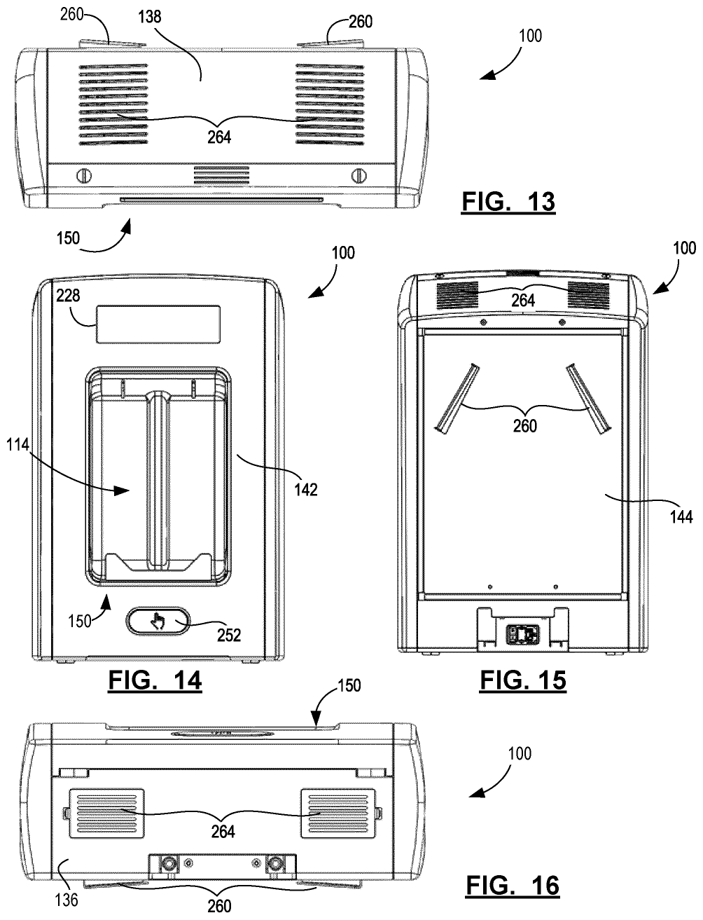

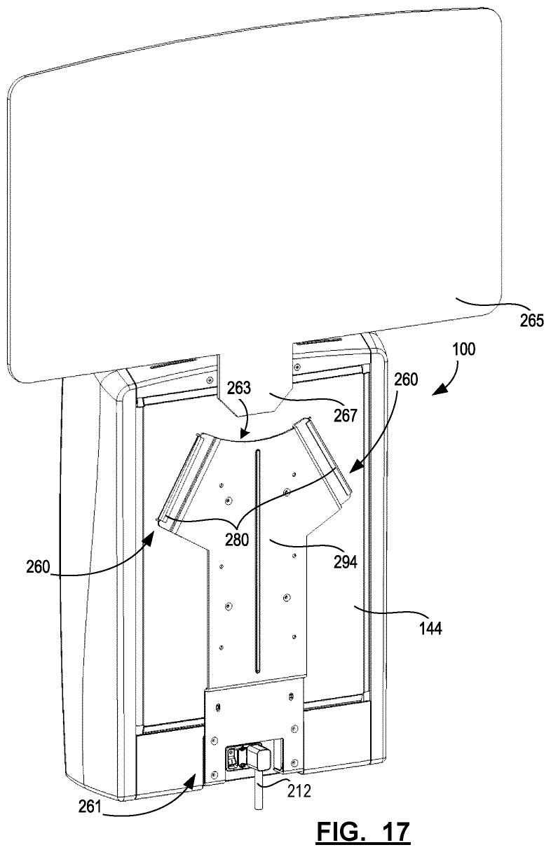

Referring to FIGS. 12 to 15, the cycle initiation toggle 250 is a button 252 in some examples. In the illustrated example, the button 252 is positioned below the access opening 150 (as in the example of FIG. 5), although it will be appreciated that in other examples one or more buttons may also or alternatively be provided at other locations.

As indicated above, the cycle initiation toggle 250 may be a multifunction toggle that can be used to initiate more than one action. The action that is initiated by a triggering of the toggle may depend on the state the toggle is in, which may depend on the state the ultraviolet treatment device 100 is in. For example, if the ultraviolet treatment device 100 is running a cycle (e.g., the ultraviolet source 230 of the device 100 is on and/or the door 190 is closed), the cycle initiation toggle 250 may be configured (e.g., by a control system, such as the control system 210) to turn off the cycle (e.g., turn off the ultraviolet source 230) if triggered. If the ultraviolet treatment device 100 is available but not running a cycle, the cycle initiation toggle 250 may be configured to initiate a cycle (e.g., close the door 190 and turn on the ultraviolet source 230) in response to being triggered.

The state of the cycle initiation toggle 250 may be indicated by the display configuration of the toggle 250 (e.g., the on/off configuration of lights, or the color or blinking of lights). FIG. 12 illustrates a button 252. The state of the button 252 may be indicated by the display configuration. The display configuration of the button 252 may include an illumination colour of one or both of an icon 254 and a border 256. The icon 254 and/or the border 256 may be illuminated one of white for standby, green for ready or start, blue for in progress, and red for error or stop. For example, the border 256 may be illuminated blue to indicate that a cycle is in progress while the icon 254 is illuminated red to indicate that triggering the button 252 will stop the cycle. In some examples, the state of cycle initiation toggle 250 is indicated by a combination of the display configuration and the display configuration of another element of the ultraviolet treatment device 100 (e.g., an on/off status, a color, or blinking status of interior visible lights).

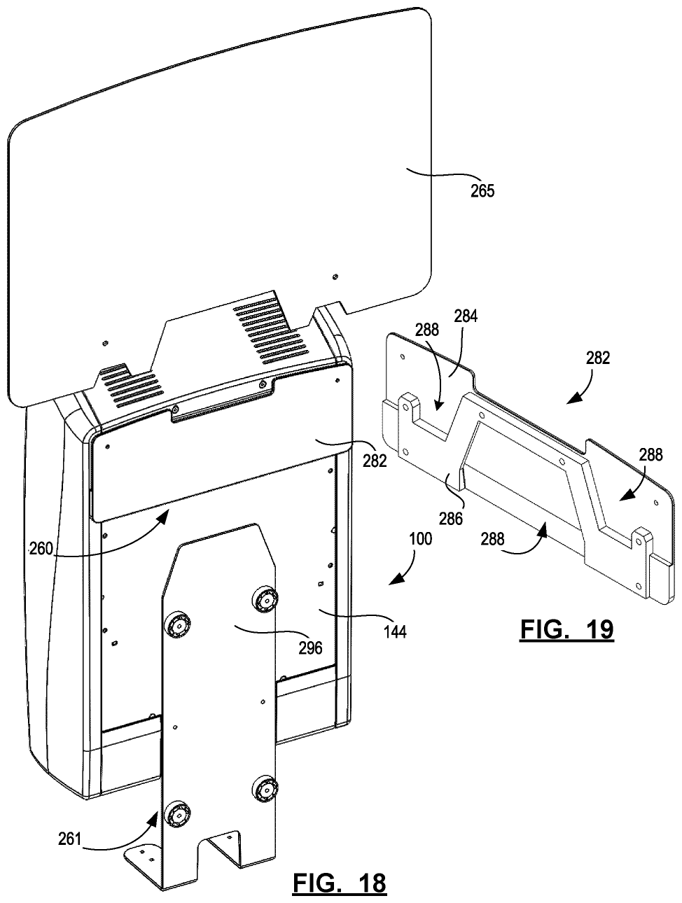

Referring to FIGS. 13 to 16, the ultraviolet treatment device 100 may include one or more brackets 260 for mounting the ultraviolet treatment device 100 in an environment (e.g., a room). It will be appreciated that in some examples the ultraviolet treatment device 100 may be positioned in an environment without the brackets (e.g., by simply being rested on a shelf or other surface) and/or may not include the brackets 260. However, one or more brackets 260 may facilitate positioning the device 100 in a user-friendly way. For example, one or more brackets 260 on the rear panel of the housing 110, as in the illustrated example, may allow the ultraviolet treatment device 100 to be secured to a vertical wall. The one or more brackets 260 may be arranged to allow the device 100 to be mounted in an environment without blocking vents 264.

The one or more brackets 260 may be arranged to allow the device 100 to be mounted in the environment such that the power coupling 212 is captured by the wall to add strain relief and prevent it from being unplugged. For example, the power coupling 212 may be received in a pocket at the rear of the device 100 such that the rear of the device can be rested against an environmental wall (e.g., when mounted to the environmental wall via one or more brackets 260) with the power coupling 212 held in position in the pocket by the environmental wall, as may be seen also from FIG. 17.

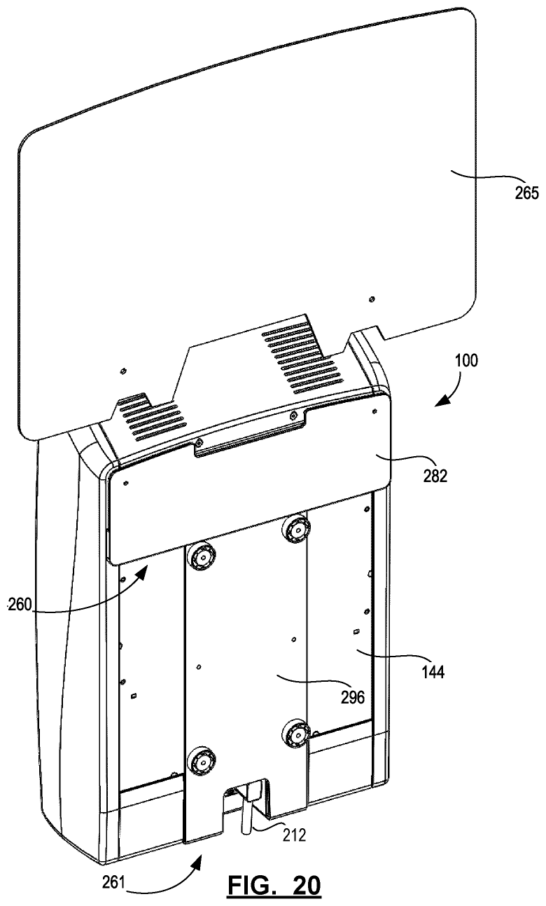

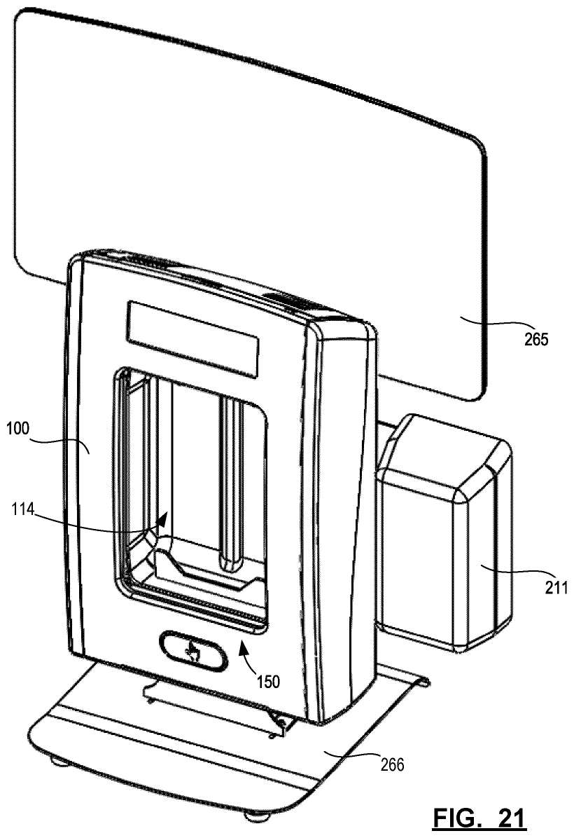

As illustrated in FIGS. 13 to 16, the one or more brackets 260 may include a hook bracket, such as the illustrated pair of angled and elongated hook brackets 280. The hook brackets 280 each include a hooked flange to rest on or over another body. However, it will be appreciated that other brackets 260 may also or alternatively be used. As illustrated in FIGS. 18 to 20, the one or more brackets 260 may include a pocket bracket, such as the single pocket bracket 282. The pocket bracket 282 includes a plate 284 and wall structure 286 forming one or more pockets 288 together with the back of the ultraviolet treatment device 100 when the bracket 282 is secured to the device 100.

Referring now to FIGS. 17 to 20, in some examples the device 100 includes and/or is secured to a mounting plate 261. The mounting plate 261 may interface with the one or more brackets 260 and be received in the one or more brackets 260 (e.g., the brackets may rest on the mounting plate 261 for greater stability) in addition to or in alternative to securing the mounting plate 261 to the rear of the housing via removable fasteners such as threaded fasteners. The mounting plate 261 may be provided to more easily mount the device 100 in the environment, and the plate 261 may include apertures 290 and/or flanges or projections 292 for use in securing the plate 261 to an environmental surface (e.g., a wall) or a stand.

In some embodiments, as exemplified in FIG. 17, the mounting plate 261 is a pocket plate 294 which includes or forms (e.g., in cooperation with the housing) a pocket 263 shaped to receive a tab or projection of a signage panel 265 to hold the signage panel adjacent the device 100. The signage panel 265 may be held above the device 100, as illustrated. The signage panel 265 may have a tab or projection 267 on a lower end of the signage panel 265 to be received in the pocket 263.

It will be apricated that the mounting plate 261 may be of any suitable form. As exemplified in FIGS. 18 to 20, the mounting plate 261 is a flat plate 296 (i.e., not forming a pocket). The flat plate 296 may be received into a pocket 288 of a pocket bracket 282. As exemplified in FIG. 19, the pocket bracket 282 may form separate pockets for the mounting plate 261 and for the signage panel 265.

The mounting plate 261 may also be used to hold another device adjacent the treatment device 100. For example, a hygiene dispenser 211 may be mounted to the mounting plate 261 on the back of the device 100 (e.g., via an arm or mount 273 mounted in a track of the plate 261). As illustrated in, e.g., FIG. 23, the hygiene dispenser 211 may be mounted to one side or the other of the treatment device 100.

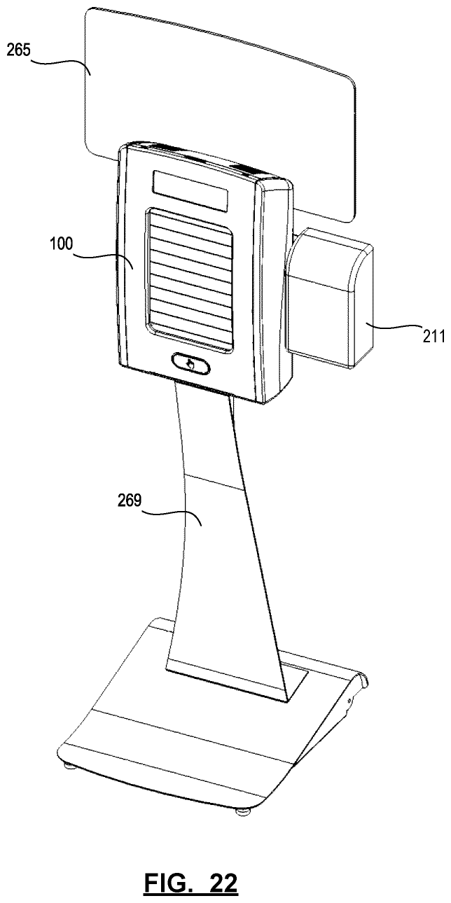

Referring to FIG. 21, the ultraviolet treatment device 100 may be secured to and/or resting on a counter or other shelf. A table stand 266 may be provided with the ultraviolet treatment device 100. The device 100 may be mounted on the table stand 266 by way of the same mounting plate 261 (e.g., bolted to the mounting plate 261 and/or the mounting plate 261 resting on the stand 266) and/or one or more brackets 260 (e.g., the one or more brackets 260 resting on the stand 266) that may be used to mount the device 100 to a wall. The table stand 266 may be shaped to elevate the ultraviolet treatment device 100 when it is resting on a surface (e.g., a table or shelf) so that the lower vents 264 remain open and/or the device 100 is stable (e.g., inhibiting the device 100 from tipping over).

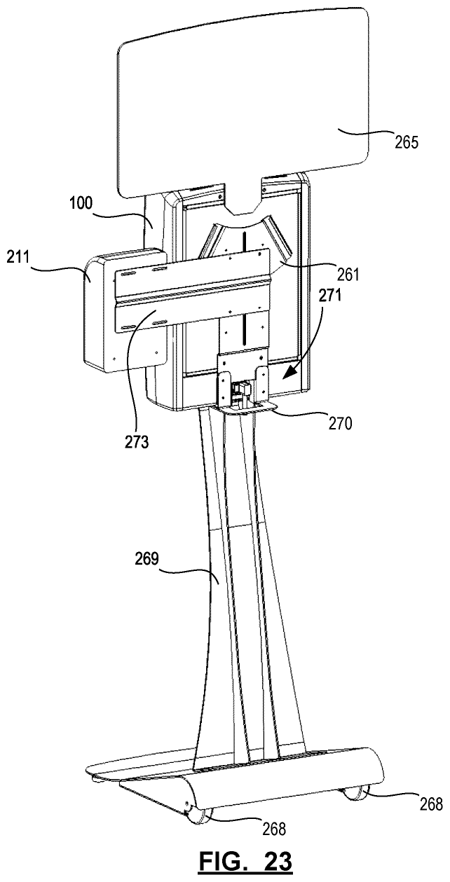

Referring now to FIGS. 22 and 23, the treatment device 100 may be mounted on a floor stand 269. The device 100 may be mounted on the floor stand 269 by way of the same plate 261 (e.g., bolted to the mounting plate 261 and/or the mounting plate 261 resting on the stand 266) and/or the one or more brackets 260 (e.g., the one or more brackets 260 resting on the stand 266) that may be used to mount the device 100 to a wall. As illustrated in FIG. 23, the floor stand 269 may be mounted to a lower portion 271 of the plate 261 (e.g., via removable threaded fasteners). The floor stand 269 may be shaped to elevate the ultraviolet treatment device 100 at a convenient height (e.g., more than half a meter) and/or keep the device 100 stable.

The floor stand 269 may include wheels 268 and/or a handle 270 to allow for easy repositioning. The wheels 268 may be positioned at a rear of the stand such that the user may tilt the stand back and then wheel it to a new position. The front of the stand does not include wheel, to provide for greater stability when not tilted.

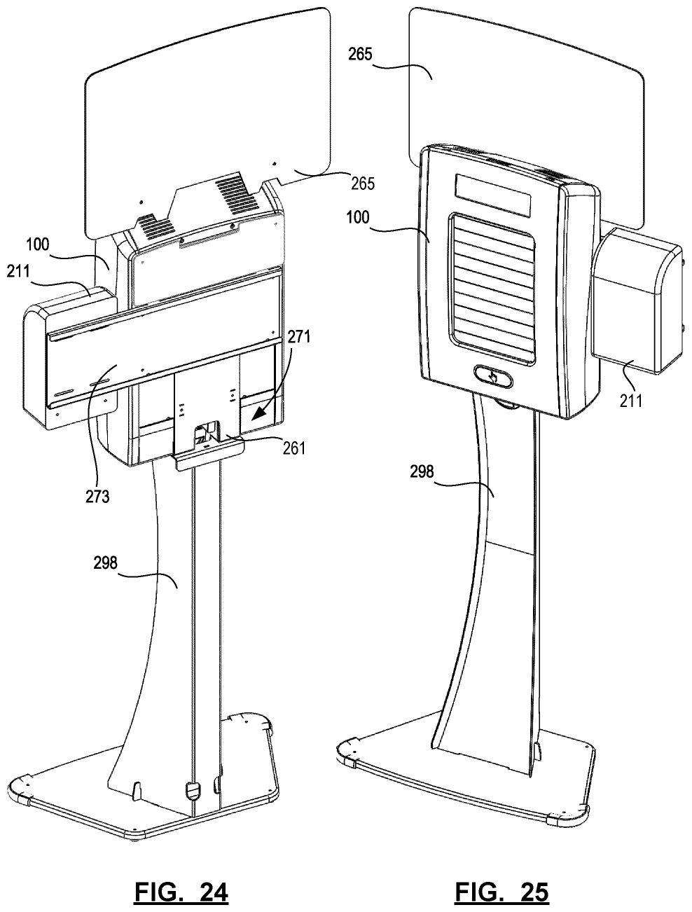

Referring now to FIGS. 24 and 25, the treatment device 100 may be mounted to a lean stand 298. The lean stand 298 may be positioned against a wall to provide stability. The lean stand includes a thick and/or heavy base plate 299 to act as a ballast to shift a center of mass of the device 100 and stand 298 forward. The baseplate 299 may keep the device 100 and stand 298 from toppling when free standing. Configuring the stand 298 to lean against an environmental wall may allow for appropriate standards of stability to be met without including all of the features of a floor stand.

The device 100 may be mounted on the lean stand 298 by way of the same plate 261 (e.g., bolted to the mounting plate 261 and/or the mounting plate 261 resting on the stand 266) and/or the one or more brackets 260 (e.g., the one or more brackets 260 resting on the stand 266) that may be used to mount the device 100 to a wall. As illustrated in FIG. 24, the lean stand 298 may be mounted to a lower portion 271 of the plate 261 (e.g., via removable threaded fasteners). The lean stand 298 may be shaped to elevate the ultraviolet treatment device 100 at a convenient height (e.g., more than half a meter) and/or keep the device 100 stable.

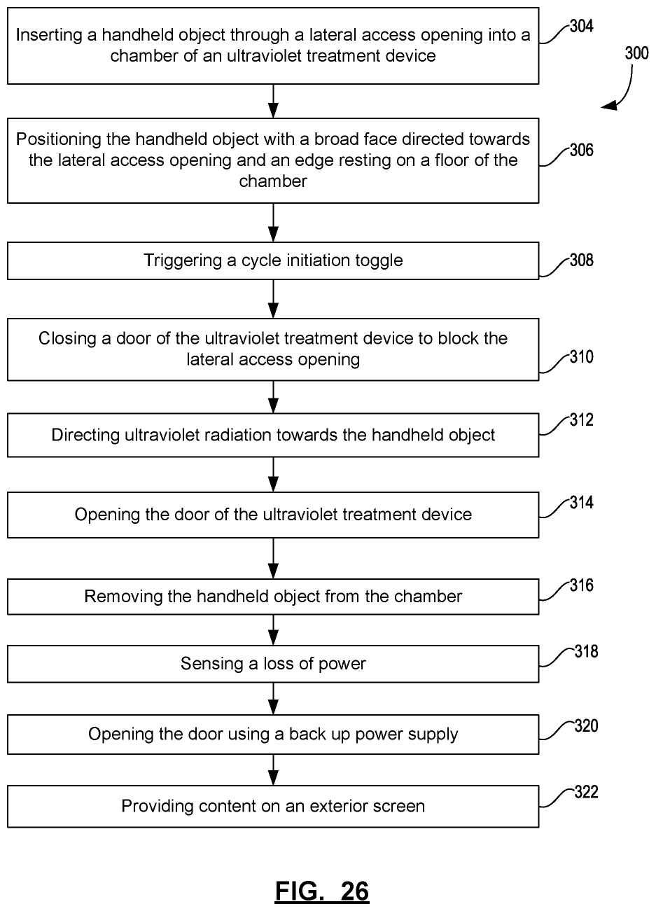

Referring now to FIG. 26, a method 300 of treating a handheld object (e.g., object 154) is shown. The method 300 includes, at step 304, inserting the handheld object (e.g., a personal computing device) into a chamber (e.g., chamber 114) of an ultraviolet treatment device (e.g., device 100) through a lateral access opening (e.g., opening 150).

At step 306, method 300 includes positioning the handheld object in the chamber with a broad planar surface of the handheld object directed towards the lateral access opening and a lower edge of the handheld device resting on a floor (e.g., floor 136) of the chamber. In some examples, step 306 includes arranging the lower edge against a retention feature or member (e.g., retention feature 170) on the floor of the chamber or on the lateral wall, the retention feature shaped to inhibit sliding of the lower edge along the floor towards the lateral access opening. It will be appreciated that one or more handheld computing devices may be treated alone, or a user may also insert one or more other objects to be cleaned (e.g., a stethoscope or a mask).

Method 300 may include a step 308 of triggering a cycle initiation toggle of the ultraviolet treatment device to initiate a cycle (e.g., the cycle initiation toggle may be a button, such as a multi-function button which is operable to start the cycle if the cycle has not begun and is operable to stop the cycle if the cycle is in progress; activating a hygiene dispenser; and/or after a countdown timer has elapsed). Step 308 is after step 306.

In some examples, method 300 includes, at step 310, closing a door of the ultraviolet treatment device to block the lateral access opening by lowering the door (e.g., in response to the triggering of the cycle initiation toggle at step 308). Lowering the door may include drawing the door down out of a pocket of a housing of the ultraviolet treatment device (e.g., if the door is a segmented door or includes a flexible panel). Step 310 may be after step 308.

Method 300 includes, at step 312, directing ultraviolet radiation towards the handheld object from an ultraviolet radiation source (e.g., source 230) of the ultraviolet treatment device. Step 312 may be carried out while the handheld object is in the chamber with the broad planar surface of the handheld object directed towards the lateral access opening and the lower edge of the handheld object resting on the floor. Step 312 may be after the step 308 of triggering a cycle initiation toggle. Step 310 and/or step 312 may be after step 308. In some examples, step 310 may be before step 308 or concurrent (e.g., if the cycle is manually initiated by closing the door). Step 312 may continue for a predetermined time and/or dosage. For example, step 312 may continue until sources of radiation have emitted a predetermined amount of radiation and/or a sensor in the chamber detects that a predetermined dosage has been received.

In some examples, method 300 includes, at step 314, opening a door of the ultraviolet treatment device to clear the lateral access opening by lifting the door (e.g., opening the door automatically after directing ultraviolet radiation towards the handheld object, without requiring further user interaction). Lifting the door may include moving the door into a pocket of a housing of the ultraviolet treatment device. Step 314 may be after step 312.

Method 300 includes, at step 316, removing the handheld object from the chamber after directing ultraviolet radiation towards the handheld object. Step 316 may be after step 314 and/or step 312.

The method 300 may include, at step 318, sensing a loss of power from a main power source (e.g., a loss of power received through a power coupling to a residential power source). The method 300 may also include, at step 320, responding to the sensed loss of power by opening the door using power stored in a backup power supply (i.e., the door may be electrically biased to open if the ultraviolet treatment device ceases to receive the power from the main power source). Step 318 may follow any step between step 308 and step 314.

The method may include, at step 322, providing content on an exterior screen of the ultraviolet treatment device. Content could include the display of instructional or advertisement content to the user, and could be displayed before or after the triggering of a cycle (e.g., displaying advertising and/or encouragement to sanitize hands to a user who needs to remain nearby anyway). Step 322 may be after and/or concurrent with any other step.

The method 300 or a subset of the steps of the method 300 (e.g., steps 310 to 312, steps 310 to 314, or steps 310 to 314 and 322) may be a cycle of a treatment device (e.g., device 100).

The present invention has been described here by way of example only. Various modification and variations may be made to these exemplary embodiments without departing from the scope of the invention, which is limited only by the appended claims.

1-54. (canceled)

55. An ultraviolet (UV) treatment device comprising:
a housing comprising
first and second pairs of walls defining a chamber therebetween, the chamber having a chamber height along a vertical axis, a chamber width along a transverse axis, and a chamber depth along a longitudinal axis, the chamber depth being less than the chamber width and less than the chamber height,
a lateral wall having a lateral access opening to the chamber, the lateral access opening having an opening width greater than the chamber depth, and
a chamber floor extending from the lateral wall and to receive a handheld device,
a retention feature configured to inhibit sliding of the handheld device along the chamber floor towards the lateral access opening when the handheld device is resting with a lower edge resting on the chamber floor, the retention feature being transparent to UV radiation; and
a UV radiation source mounted to the housing and configured to direct UV radiation into the chamber.

56. The UV treatment device of claim 55 wherein the retention feature comprises one of a projection from the chamber floor, a projection from the lateral wall, a sloped portion of the chamber floor sloping down away from the lateral access opening, a groove in the chamber floor, a high-friction surface of the chamber floor, and a coarse surface of the chamber floor.

57. The UV treatment device of claim 55 wherein the chamber has a rectangular horizontal cross-section; and wherein each of the first pair of lateral walls has a width less than a width of each of the second pair of lateral walls.

58. The UV treatment device of claim 55 wherein the housing includes a door moveable between a closed position blocking the lateral access opening and an open position removed from the lateral access opening to provide access to the chamber through the lateral access opening for a user to place the handheld device within the chamber with the lower edge resting on the chamber floor.

59. The UV treatment device of claim 58 wherein the housing defines a recess for receiving the door when in the open position.

60. The UV treatment device of claim 58 wherein the door comprises a segmented door.

61. The UV treatment device of claim 58 wherein the door comprises a flexible panel.

62. The UV treatment device of claim 58 further comprising a door control system configured to control positioning of the door, the door control system configured to cause the door to be in the open position when the UV radiation source is not activated.

63. The UV treatment device of claim 62 further comprising a visible light source configured to illuminate the chamber when the door is in the closed position.

64. The UV treatment device of claim 55 further comprising a cycle initiation toggle for initiating a UV cycle.

65. The UV treatment device of claim 64 wherein the cycle initiation toggle comprises one of a button, a light sensor, a camera, an audio sensor, a touch screen, a door position sensor, and a hygiene dispenser.

66. The UV treatment device of claim 62 wherein the door control system is configured to receive power from a power source; and wherein the door control system is configured to cause the door to be biased to the open position when there is no power from the power source.

67. The UV treatment device of claim 66 wherein the UV treatment device includes an energy storage device coupled to the power source to detect a loss of power from the power source and coupled to the door control system to open the door in response to detecting the loss of power from the power source.

68. The UV treatment device of claim 55 further comprising a screen carried by the housing and configured to provide content to a user during a cycle.

69. The UV treatment device of claim 62 wherein the door control system is configured to open the door automatically at an end of a cycle.

70. The UV treatment device of claim 55 wherein the housing includes a transparent internal wall separating the chamber from the UV radiation source; and wherein the transparent internal wall includes a visibility-enhancing pattern.

71. A method of treating a handheld device, the handheld device having a broad planar surface and a lower edge adjacent to the broad planar surface, the method comprising:
inserting the handheld device into a chamber of an ultraviolet (UV) radiation treatment device through a lateral access opening;
positioning the handheld device in the chamber with the broad planar surface of the handheld device directed towards the lateral access opening and the lower edge of the handheld device resting on a floor of the chamber;
directing, while the handheld device is in the chamber with the broad planar surface of the handheld device directed towards the lateral access opening and the lower edge of the handheld device resting on the floor, UV radiation towards the handheld device from an UV radiation source of a UV treatment device; and
removing the handheld device from the chamber after directing UV radiation towards the handheld device.

72. The method of claim 71 further comprising, between positioning the handheld device in the chamber and directing UV radiation towards the handheld device, closing a door of the UV treatment device to block the lateral access opening.

73. The method of claim 72 closing the door includes drawing the door down out of a recess of a housing of the UV treatment device.

74. The method of claim 72 further comprising between directing UV radiation towards the handheld device and removing the handheld device from the chamber, opening the door of the UV treatment device to clear the lateral access opening by lifting the door.
